(19) United States
(12) Patent Application Publication
(10) Pub. No.: US 2024/0285683 A1
(43) Pub. Date: Aug. 29, 2024

(54) Compositions And Methods For Enhancing Immunotherapy

(71) Applicant: The Trustees of Princeton University, Princeton, NJ (US)

(72) Inventor: Joshua D. Rabinowitz, Princeton, NJ (US)

(21) Appl. No.: 18/413,717

(22) Filed: Jan. 16, 2024

Related U.S. Application Data
(63) Continuation of application No. 16/621,660, filed on Dec. 11, 2019.
(60) Provisional application No. 62/619,376, filed on Jan. 19, 2018.
(60) Provisional application No. 62/525,357, filed on Jun. 27, 2017.

(51) Int. Cl. A61K 35/17; C07K 16/28; C07K 16/30; C12N 5/0783; C12N 9/02; C12N 15/113

(52) U.S. Cl. CPC A61K 35/17; C07K 16/2818; C07K 16/3084; C12N 5/0636; C12N 9/0036; C12N 15/1138; C12N 2310/14; C12N 2500/02; C12N 2501/71

(57) ABSTRACT

The present invention provides, in some embodiments, methods of promoting an immune response in a subject in need thereof, comprising administering to a subject a population of immune cells that express an exogenous enzyme that facilitates immune cell function in a nutrient-poor environment. Other embodiments of the invention include methods of promoting an immune response to a tumor in a subject in need thereof, comprising administering to the subject an effective amount of an agent that provides a one-carbon unit (e.g., formate) and an agent that promotes an anti-tumor response, and methods of promoting an immune response to a tumor in a subject in need thereof, comprising administering to a subject an effective amount of an agent that inhibits consumption of metabolic fuels by tumor cells. The invention also provides, in other embodiments, compositions comprising an ex vivo population of immune cells expressing an exogenous enzyme that enhances immune cell function in nutrient poor environments, and compositions comprising a nucleic acid expression construct encoding an inhibitor of glucose metabolism, and a pharmaceutically acceptable carrier or excipient.

### RELATED APPLICATIONS

This application is a continuation of U.S. application Ser. No. 16/621,660, filed on Dec. 11, 2019, which is the U.S. National Stage of International Application No. PCT/US2018/039817, filed on Jun. 27, 2018, published in English, which claims the benefit of U.S. Provisional Application No. 62/525,357, filed Jun. 27, 2017, and U.S. Provisional Application No. 62/619,376, filed Jan. 19, 2018. The entire teachings of these applications are incorporated herein by reference.

### STATEMENT OF GOVERNMENT SUPPORT

This invention was made with government support under Grant No. DK113643 and Grant No. CA163591 awarded by the National Institutes of Health. The government has certain rights in the invention.

### BACKGROUND

Metabolic factors can inhibit immune responses. For example, immune cells need a myriad of small molecules, such as glucose, glutamine, arginine, tryptophan, and other nutrients and metabolites to proliferate and to fight infection. When one or more of these nutrients is in short supply, immune response can be limited. In addition, certain metabolites may tend to skew or suppress immune responses in a manner that is disadvantageous to the patient. For example, kynurenine and adenosine are endogenous immunosuppressive metabolites that may suppress immune responses to infections and/or tumors. Lactate is another metabolite that may favor less aggressive immune responses, and high lactate in tumors may impair cancer immunotherapy, especially in poorly perfused regions of solid tumors where lactate accumulates. A particular need of immune cells, which is shared also with cancer cells, is oxidized nicotinamide adenine dinucleotide (NAD) and oxidized carbon for use in synthesis of amino acids and nucleotides. Such oxidized cofactors and carbon may be in particular short supply in the tumor microenvironment, due to poor perfusion and low O₂. Thus, there is a need for technologies that enable more effective immune response in nutrient-limited environments, including environments limited for oxidized NAD and oxidized carbon.

In addition, activation of immune cells, such as T cells, requires the availability of metabolic fuels, such as glucose. The fate of T cell activation can be dictated by the environmental availability of glucose, and the ratio of glucose to other fuels such as lactate. The tumor microenvironment is typically poor in glucose and high in lactate (see, e.g., Kamphorst, J. J, et al., Human pancreatic cancer tumors are nutrient poor and tumor cells actively scavenge extracellular protein. Cancer Research 75(3): 544-553(2015)), which can create a barrier to immune cell activation in and around the tumor and thus reduce the efficacy of cancer immunotherapy, especially for solid tumors. Accordingly, there is a need for compositions and methods that can effectively alter the metabolic composition of a tumor microenvironment to better support immune cell activation and enhance anti-tumor immune responses in cancer patients.

### SUMMARY OF THE INVENTION

The present invention provides, in an embodiment, a method of promoting an immune response (e.g., a T cell response, an antitumor immune response) in a subject in need thereof, comprising administering to a subject a population of immune cells that express an exogenous enzyme (e.g., NADH oxidase) that catalyzes the oxidation of nicotinamide adenine dinucleotide, reduced form (NADH) to nicotinamide adenine dinucleotide, oxidized form (NAD⁺) (e.g., using molecular oxygen as the electron acceptor).

In another embodiment, the invention provides a composition comprising an ex vivo population of immune cells expressing an exogenous enzyme that catalyzes the oxidation of NADH.

In yet another embodiment, the invention provides a method of promoting (e.g., enhancing) an immune response (e.g., to a tumor) in a subject in need thereof. The method comprises the step of administering to a subject an agent that inhibits consumption of metabolic fuels by tumor cells, or a nucleic acid encoding an agent that inhibits consumption of metabolic fuels by tumor cells. In a particular embodiment, the agent (e.g., shRNA) is an inhibitor of glucose metabolism (e.g., an inhibitor of GLUT1 and/or GLUT3).

In another embodiment, the invention provides a composition comprising a nucleic acid expression construct encoding an inhibitor of glucose metabolism, and a pharmaceutically-acceptable carrier or excipient. In a particular embodiment, the nucleic acid expression construct encodes an inhibitor of a glucose transporter (e.g., an inhibitor of GLUT1 and/or GLUT3).

In another embodiment, the invention provides a method of promoting an immune response to a tumor in a subject in need thereof, comprising administering to the subject an effective amount of an agent that provides a one-carbon unit and an agent that promotes an anti-tumor response.

In another embodiment, the invention provides a method of treating cancer in a subject in need thereof, comprising administering to the subject an effective amount of an agent that provides a one-carbon unit and an agent that promotes an immune (e.g., anti-tumor) response.

In another embodiment, the invention provides a method of treating immune dysfunction in a subject in need thereof, comprising administering to the subject (e.g., an aged human) an effective amount of an agent that provides a one-carbon unit and an agent that promotes an anti-tumor response.

The compositions and methods described herein are useful for increasing the availability of metabolic fuels in and surrounding a tumor, thereby creating a more favorable environment for immune cell activation to enhance anti-tumor immune responses, including in combination with other agents, such as PD-1, PD-1L, or CTLA-4 checkpoint inhibitors. The compositions and methods described herein, in certain embodiments, are also useful for improving the efficacy of immunotherapy methods, including CAR-T therapy.

### BRIEF DESCRIPTION OF THE DRAWINGS

The foregoing will be apparent from the following more particular description of example embodiments.

FIGS. 1A-1E are line graphs of tumor volume (mm³) versus time (days), and show the individual tumor growth trajectories of subcutaneous CT26 tumors on female BALB/c mice receiving no treatment (FIG. 1A) or treated with 20 mg/mL formate (FIG. 1), anti-PD-1 (FIG. 1C), anti-PD-1 and 20 mg/mL formate (FIG. 1D), or anti-PD-1 and anti-CTLA4 (FIG. 1E).

FIG. 2A is a Kaplan-Meier plot, and shows the Kaplan-Meier survival data of the mice from the experiments depicted in FIGS. 1A-1E (CCD1=formate). The lines in the graph follow the order of the groups in the key.

FIG. 2B is a line graph of tumor volume (mm³) versus time (days), and shows the mean tumor volume of the mice from the experiments depicted in FIGS. 1A-1E (CCD1=formate). The lines in the graph follow the order of the groups in the key.

FIG. 3A is a bar graph of percent labeled acetyl coenzyme A (CoA) in non-transduced (NTD) CAR-T cells and CAR-T cells expressing CD28ζ or CD28ζ and NADPH oxidase (NOX), and shows that CAR-T cells intrinsically actively metabolize lactate.

FIG. 3B is a bar graph of percent labeled β-hydroxy-β-methylglutaryl (HMG) CoA in NTD CAR-T cells and CAR-T cells expressing CD28ζ or CD28ζ and NADPH oxidase (NOX), and shows that CAR-T cells intrinsically actively metabolize lactate.

FIG. 4 is a line graph of oxygen consumption (pmoles/minute) versus time (minutes), and shows that cytosolic NOX drives oxygen consumption and NAD production in CAR-T cells comprising a CAR targeting mesothelin.

FIG. 5 is a line graph of oxygen consumption (pmoles/minute) versus time (minutes), and shows cytosolic NOX (NOX) expression induces basal T cell oxygen consumption and mitochondrial NOX (MitoNox) expression supports oxygen consumption, especially in the presence of lactate, in CAR-T cells comprising a CAR targeting GD-2.

### DETAILED DESCRIPTION OF THE INVENTION

A description of example embodiments of the invention follows.

### Methods for Enhancing Immunotherapy

It is an object of the present invention to improve the effectiveness of immunotherapy, particularly cancer immunotherapy. The invention contemplates enhancing immune responses (e.g., T cell responses) against a target (e.g., a tumor) by creating immune cells (e.g., CAR-T cells) that are better able to cope with the metabolic environment of the target (e.g., the high lactate environment of the tumor), for example, by increasing levels of oxidized NAD and/or oxidized carbon available to immune cells (e.g., for use in synthesis of amino acids and nucleotides in vivo, and/or by creating a more favorable metabolic environment for immune cell activation, for example, by increasing the levels and availability of metabolic fuels that support immune cell activation in and surrounding a tumor. As a consequence, the levels, activation state, and/or cytotoxic capacity of immune cells, including activated T cells (e.g., CAR-T, Th1, and/or Th17 cells), in the tumor, the tumor microenvironment, or both are increased.

The present invention also contemplates ex vivo engineering of immune cells to endow them with metabolic capacity to survive, activate, proliferate, and/or carry out immune effector functions in the presence of a nutrient-limited microenvironment (e.g., tumor microenvironment), such as by expressing one or more enzymes that produce an increase in the level of oxidized NAD and/or an increase in the level of oxidized carbon (e.g., pyruvate) in the immune cells, for example, by expressing one or more enzymes that catalyze the reaction of NADH and molecular oxygen to yield water or hydrogen peroxide. In certain embodiments, the activity of such an enzyme may also limit tumor growth, for example, by consuming molecular oxygen and thereby limiting its availability to tumor cells.

The present invention further contemplates creating a more favorable metabolic environment for immune cell activation by employing agents that contribute to one or more of the following outcomes: an increase in the level of glucose, a decrease in the level of lactate, an increase in the level of proteogenic amino acids, a decrease in the level of amino acid degradation products, or an increase in usable 1-carbon units, in or around a tumor in a subject. The present invention also contemplates creating a more favorable metabolic environment for immune cell activation by employing agents that contribute to one or more of the following outcomes: an increase in the level of glucose, a decrease in the level of lactate, an increase in the level of proteogenic amino acids, a decrease in the level of amino acid degradation products, or an increase in usable 1-carbon units, in a subject receiving a vaccine or in a subject suffering from an infection.

Accordingly, in various embodiments, the invention relates to a method of promoting an immune response in a subject in need thereof. In certain embodiments, the invention relates to a method of promoting an immune response in a subject in need thereof that comprises administering to a subject an exogenous enzyme (e.g., NADH oxidase) that catalyzes the oxidation of NADH to NAD⁺ in immune cells in the subject. In some embodiments, a population of immune cells that express an exogenous enzyme that catalyzes the oxidation NADH to NAD⁺ is administered to the subject. In some embodiments, the immune cells comprise or consist essentially of CAR-T cells.

In a particular embodiment, the exogenous enzyme is an NADH oxidase (NOX). The NADH oxidase can be naturally occurring or non-naturally occurring (e.g., engineered). The NADH oxidase can be isolated (e.g., from a natural source), recombinant or synthetic. Examples of NADH oxidases from a variety of organisms that are suitable for use in the methods and compositions described herein are known in the art. In some embodiments, the NADH oxidase uses oxygen (O₂) as an electron acceptor. In some embodiments, the NADH oxidase catalyzes reaction of NADH and O₂ into water (H₂O). In some embodiments, the NADH oxidase catalyzes the reaction of NADH and O₂ into H₂O₂. In a particular embodiment, the NADH oxidase is an NADH oxidase from Lactobacillus brevis (LbNOX) (UniProtKB Accession Number Q8KRG4). In a particular embodiment, the NADH oxidase is an NADH oxidase from Amphibacillus xylanus (see Niimura, Y., et al., Journal of Bacteriology 182(18): 5046-5051 (2000), the contents of which are incorporated by reference herein in their entirety).

Examples of other NADH oxidases that are suitable for use in the methods and compositions of the invention include variants of naturally occurring NADH oxidases (e.g., variants having at least about 80%, about 85%, about 90%, about 95%, about 98%, or about 99% amino acid sequence identity to a naturally occurring NADH oxidase, such as a naturally occurring (e.g., wild-type) NADH oxidase from Lactobacillus brevis. In some embodiments, variants of naturally occurring NADH oxidases include enzymes that have been engineered to have reduced immunogenicity in a host organism (e.g., a human subject). Methods of engineering proteins (e.g., enzymes) for reduced immunogenicity in a host organism are well-known in the art. In some embodiments, the NADH oxidase sequence has been codon optimized to enhance protein expression.

As used herein, the term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. The sequence identity comparison can be examined throughout the entire length of a given protein, or within a desired fragment of a given protein. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

NADH oxidases can be unmodified or modified (e.g., post-translationally modified), and/or unlabeled or labeled (e.g., with a detectable label, such as a fluorophore or hapten). In certain embodiments, an NADH oxidase is coupled (e.g., covalently linked) to one or more additional molecules (e.g., an enzyme that converts lactate to pyruvate, a cytotoxic agent). In a particular embodiment, the NADH oxidase is coupled to a lactate dehydrogenase enzyme. In certain embodiments, the NADH oxidase is co-expressed with an enzyme (e.g., catalase) to convert hydrogen peroxide (H₂O₂) made from the NADH oxidase into water.

In some embodiments, the exogenous enzyme is a lactate oxidase enzyme. A lactate oxidase enzyme uses oxygen to oxidize lactate to pyruvate.

An exogenous NADH oxidase and/or other desired protein(s) can be introduced into immune cells as a protein, or as a nucleic acid molecule that encodes the NADH oxidase or other protein, using well-known techniques, including any of the various techniques described herein. In a particular embodiment, an exogenous NADH oxidase is introduced (e.g., transfected) into immune cells as a nucleic acid molecule that encodes the NADH oxidase. Suitable nucleic acid constructs for introduction into cells are known in the art and include the various nucleic acid constructs described herein. In an embodiment, the nucleic acid molecule that encodes the NADH oxidase is a DNA expression vector (e.g., a viral vector, a non-viral vector).

In some embodiments, an NADH oxidase and/or other desired protein(s) is selectively expressed in mitochondria of the immune cells. An advantage of mitochondrial expression of an NADH oxidase enzyme is that mitochondria are the physiological site of oxygen-dependent NADH oxidation, and accordingly, expression of NADH oxidase in mitochondria is expected to avoid physiological perturbations to the cytosolic NADH pool and retain regulation of the cytosolic NADH/NAD ratio by electron transport into mitochondria. Moreover, mitochondria are the physiological site for conversion of pyruvate to oxaloacetate, a key precursor for aspartate.

In some embodiments, an NADH oxidase and/or other desired protein(s) is selectively expressed in the cytosol of the immune cells. An advantage of cytosolic expression of an NADH oxidase enzyme is expected to be the ability to directly produce cytosolic NADH and oxidized carbon without the need for electron transport into mitochondria, enabling conversion of exogenous (e.g., circulating or microenvironmental) lactate into pyruvate without the need for electron transport into mitochondria.

In certain embodiments, the exogenous NADH oxidase and/or other desired protein(s) (e.g., NADH oxidase), or an encoding nucleic acid molecule, is introduced (e.g., transfected) into immune cells ex vivo (e.g., into an ex vivo population of immune cells). In a particular embodiment, the exogenous NADH oxidase and/or other desired protein(s) (e.g., NADH oxidase), or an encoding nucleic acid molecule, is introduced into a population of T cells. In some embodiments, the T cells are chimeric antigen receptor T cells (CAR-T cells). CARs are artificial receptors that are engineered to contain an immunoglobulin antigen binding domain, such as a single-chain variable fragment (scFv). A CAR may, for example, comprise an scFv fused to a TCR CD3 transmembrane region and endodomain. An scFv is a fusion protein of the variable regions of the heavy (V_{H}) and light (V_{L}) chains of immunoglobulins, which may be connected with a short linker peptide of approximately 10 to 25 amino acids (Huston J. S. et al. Proc Natl Acad Sci USA 1988; 85(16):5879-5883). The linker may be glycine-rich for flexibility, and serine or threonine rich for solubility, and may connect the N-terminus of the V_{H} to the C-terminus of the V_{L}, or vice versa. The scFv may be preceded by a signal peptide to direct the protein to the endoplasmic reticulum, and subsequently the T cell surface. In the CAR, the scFv may be fused to a TCR transmembrane and endodomain. A flexible spacer may be included between the scFv and the TCR transmembrane domain to allow for variable orientation and antigen binding. The endodomain is the functional signal-transmitting domain of the receptor. An endodomain of a CAR may comprise, for example, intracellular signalling domains from the CD3 (-chain, or from receptors such as CD28, 41BB, or ICOS. A CAR may comprise multiple signalling domains, for example, but not limited to, CD3z-CD28-41BB or CD3z-CD28-OX40.

The CAR-T cells can be designed to recognize an antigen(s) on tumor cells. Tumor antigens expressed by cancer cells may include, for example, cancer-testis (CT) antigens encoded by cancer-germ line genes, such as MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE and immunogenic fragments thereof (Simpson et al. Nature Rev (2005) 5, 615-625, Gure et al., Clin Cancer Res (2005) 11, 8055-8062; Velazquez et al., Cancer Immun (2007) 7, 11; Andrade et al., Cancer Immun (2008) 8, 2; Tinguely et al., Cancer Science (2008); Napoletano et al., Am J of Obstet Gyn (2008) 198, 99 e91-97).

Other tumor antigens include, for example, overexpressed, upregulated or mutated proteins and differentiation antigens particularly melanocyte differentiation antigens such as p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR.alpha. fusion protein, PTPRK, K-ras, N-ras, triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (PV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68KP1, CO-029, FGF-5, G250, Ga733 (EpCAM), HTgp-175, M344, MA-50, MG7-Ag, MOV18, NB170K, NY-CO-1, RCAS 1, SDCCAG16, TA-90 (Mac-2 binding protein\cyclophilin C-associated protein), TAAL6, TAG72, TLP, TPS and tyrosinase related proteins such as TRP-1, TRP-2.

Other tumor antigens include out-of-frame peptide-MHC complexes generated by the non-AUG translation initiation mechanisms employed by “stressed” cancer cells (Malarkannan et al. Immunity 1999 June; 10(6):681-90).

Yet other tumor antigens, as well as their associated indication(s) are listed in the table below:

| Antigen | Indication | Reference |
|---|---|---|
| CD19 | B-cell malignanices | Porter et al., 2011 |
| CD20 | ″ | Rufener et al., 2016 |
| CD22 | ″ | Fry et al., 2018 |
| CD123 | AML | Ruella et al., 2016 |
| CD33 | ″ | Kenderian et al., 2016 |
| BCMA | Multiple Myeloma | Ali et al., 2016 |
| CS1 | ″ | Chu et al., 2014 |
| Kappa Light Chain | ″ | Ramos et al., 2016 |
| CD138 (Syndecan 1) | ″ | Tian t al., 2017 |
| MUC1 glycan | “Universal solid tumor antigen” | Posey et al., 2016 |
| ERBB2 | Ovarian, breast, GBM, | Liu et al., 2016 |
| | osteosarcoma | |
| Mesothelin | Pancreatic, Mesothelioma | Beatty et al., 2018 |
| Fibroblast activating protein | Mesothelioma, lung, colon, | Wang et al., 2014 |
| (FAP) | pancreatic | |
| Folate Receptor- alpha | Ovarian cancer | Kandalaft et al., 2012 |
| GD-2 | Neuroblastoma | Richman et al., 2018 |
| PSMA | Prostate cancer | Kloss et al., 2018 |
| EGFR | NSCLC, epithelial carcinoma, | Golubovskaya et al., |
| | glioma | 2018 |
| EGFRv111 | GBM | O'Rourke et al., 2017 |
| CAIX | Renal Cell carcinoma (RCC) | Larners et al., 2013 |
| CEACAM | Lung, colon, pancreatic | Burga et al., 2015 |
| CD70 | Head and neck squamous cell | Park et al., 2018 |
| | carcinoma | |
| GFRalpha4 | Thyroid | |

Other tumor antigens are well-known in the art (see for example WO00/20581; Cancer Vaccines and Immunotherapy (2000) Eds Stern, Beverley and Carroll, Cambridge University Press, Cambridge). The sequences of these tumor antigens are readily available from public databases but are also found in WO 1992/020356 A1, WO 1994/005304 A1, WO 1994/023031 A1, WO 1995/020974 A1, WO 1995/023874 A1 and WO 1996/026214 A1.

Methods of obtaining and/or preparing populations of T cells, including CAR-T cells, are known in the art. In addition, in some embodiments, the T-cells (e.g., CAR-T cells) are treated with an agent that provides a one-carbon unit (e.g., formic acid, or a prodrug thereof, or a salt of either of the foregoing), for example, by adding the agent to the cell culture medium of the T cells.

In particular embodiments, the invention relates to a method of promoting an immune response in a subject in need thereof that comprises the step of administering to a subject an agent (e.g., an effective amount of an agent) that inhibits consumption of metabolic fuels by tumor cells. In a certain embodiment, the method comprises the step of administering to a subject a nucleic acid encoding an agent that inhibits consumption of metabolic fuels by tumor cells. In particular embodiments, the method comprises the step of administering to a subject an agent (e.g., an effective amount of an agent) that is itself a metabolic fuel providing 1-carbon units for tumor-fighting immune cells, such as formate, 5-formyl-THF, serine, glycine, monomethylglycine, dimethylglycine, glycine betaine, choline, or glucose, including esters and prodrugs thereof.

One-carbon metabolism is the process by which one-carbon, or single-carbon, units are transferred from one molecule to another. Typically in one-carbon metabolism, a carbon unit is transferred from serine or glycine to tetrahydrofolate (THF) to form methylene-THF. Examples of one-carbon units include methyl (—CH₃), methylene (═CH₂), methenyl (═CH₂—), formyl (—C(O)H), formimino (—CH═NH—) and hydroxymethyl (—CH₂OH). Sources of one-carbon units include serine, glycine, histidine, tryptophan, formic acid, 5-formyl-THF, monomethylglycine, dimethylglycine, glycine betaine, choline and glucose, a prodrug (e.g., an ester prodrug, an amide prodrug) of any of the foregoing or a salt (e.g., a pharmaceutically acceptable salt) of any of the foregoing (including the foregoing sources of one-carbon units as well as their prodrugs). Sources of one-carbon units also include folic acid, 5-methyl-THF, 5-formyl-THF, a prodrug (e.g., an ester prodrug, an amide prodrug) of any of the foregoing or a salt (e.g., a pharmaceutically acceptable salt) of any of the foregoing (including the foregoing sources of one-carbon units and their prodrugs).

In particular embodiments, the method comprises the step of administering to a subject an agent (e.g., an effective amount of an agent) that provides a one-carbon unit (e.g., a source of a one-carbon unit, such as any of the sources of one-carbon units described herein). In a particular embodiment, the agent that provides a one-carbon unit is formic acid or a prodrug thereof, or a pharmaceutically acceptable salt of either of the foregoing (e.g., calcium formate).

As used herein, the term “prodrug” means a compound that can be hydrolyzed, oxidized, metabolized or otherwise react under biological conditions to provide a one-carbon unit suitable for use in one-carbon metabolism. Prodrugs may become active upon such reaction under biological conditions, or they may have activity in their unreacted forms. A prodrug may undergo reduced metabolism under physiological conditions (e.g., due to the presence of a hydrolyzable group), thereby resulting in improved circulating half-life of the prodrug (e.g., in the blood). Prodrugs can be prepared using well-known methods, such as those described by Burger's Medicinal Chemistry and Drug Discovery (1995) 172-178, 949-982 (Manfred E. Wolff ed., 5^{th} ed).

In one embodiment, the prodrug comprises a hydrolyzable group. As used herein, the term “hydrolyzable group” refers to a moiety that, when present in a molecule (e.g., an agent that provides a one-carbon unit), yields a carboxylic acid or salt thereof upon hydrolysis. An ester, for example, can be hydrolyzed to a carboxylic acid, or a salt thereof, under appropriate conditions. Hydrolysis can occur, for example, spontaneously under acidic or basic conditions in a physiological environment (e.g., blood, metabolically active tissues such as, for example, liver, kidney, lung, brain), or can be catalyzed by an enzyme(s), (e.g., esterases, peptidases, hydrolases, oxidases, dehydrogenases, lyases or ligases). A hydrolyzable group can confer upon a compound of the invention advantageous properties in vivo, such as improved water solubility, improved circulating half-life in the blood, improved uptake, improved duration of action, or improved onset of action.

In one embodiment, the hydrolyzable group does not destroy the biological activity of the compound. In an alternative embodiment, a compound with a hydrolyzable group can be biologically inactive, but can be converted in vivo to a biologically active compound.

In one embodiment, the prodrug is an ester comprising a hydrolyzable group. In one embodiment, the hydrolyzable group is selected from the group consisting of (C₁-C₁₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, aryl and aryl(C₁-C₁₀)alkyl, and is optionally substituted with 1 to 3 substituents selected from the group consisting of halo, nitro, cyano, cyclopropyl, cyclobutyl, cyclopentyl, cyclohexyl, amino, (C₁-C₆)alkylamino, di(C₁-C₆)alkylamino, (C₁-C₆)alkyl, halo(C₁-C₆)alkyl, (C₁-C₆)alkoxy, halo(C₁-C₆)alkoxy, morpholino, phenyl, and benzyl. In another embodiment, the hydrolyzable group is selected from the group consisting of methyl, ethyl, n-propyl, isopropyl, n-butyl, sec-butyl, isobutyl, tert-butyl, pentyl, hexyl, heptyl, allyl, ethoxymethyl, methoxyethyl, methoxyethoxymethyl, methoxyethoxyethyl, benzyl, pentafluorophenyl, 2-N-(morpoholino)ethyl, dimethylaminoethyl and para-methoxybenzyl. In another embodiment, the hydrolyzable group is polyethylene glycol (e.g., —(OCH₂CH₂O)ₙR, wherein n is an integer from 1 to about 100, for example, from 1 to about 50, from 1 to about 25, from 1 to about 10 or from 1 to about 5; and R is hydrogen, a second one-carbon unit, such as formyl, or (C₁-C₁₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, aryl and aryl(C₁-C₁₀)alkyl, optionally substituted with 1 to 3 substituents selected from the group consisting of halo, nitro, cyano, cyclopropyl, cyclobutyl, cyclopentyl, cyclohexyl, amino, (C₁-C₆)alkylamino, di(C₁-C₆)alkylamino, (C₁-C₆)alkyl, halo(C₁-C₆)alkyl, (C₁-C₆)alkoxy, halo(C₁-C₆)alkoxy, morpholino, phenyl, and benzyl).

In certain embodiments, a molecule (e.g., an agent that provides a one-carbon unit) comprises two or more hydrolyzable groups (e.g., two or more esters each independently comprising a hydrolyzable group). In compounds comprising two or more esters each independently comprising a hydrolyzable group, each hydrolyzable group can be independently selected from the group consisting of (C₁-C₁₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, aryl and aryl(C₁-C₁₀)alkyl, and is optionally substituted with 1 to 3 substituents selected from the group consisting of halo, nitro, cyano, cyclopropyl, cyclobutyl, cyclopentyl, cyclohexyl, amino, (C₁-C₆)alkylamino, di(C₁-C₆)alkylamino, (C₁-C₆)alkyl, halo(C₁-C₆)alkyl, (C₁-C₆)alkoxy, halo(C₁-C₆)alkoxy, morpholino, phenyl, and benzyl. In another embodiment, each hydrolyzable group is independently selected from the group consisting of methyl, ethyl, n-propyl, isopropyl, n-butyl, sec-butyl, isobutyl, tert-butyl, pentyl, hexyl, heptyl, allyl, ethoxymethyl, methoxyethyl, methoxyethoxymethyl, methoxyethoxyethyl, benzyl, pentafluorophenyl, 2-N-(morpoholino)ethyl, dimethylaminoethyl and para-methoxybenzyl.

In some embodiments, two or more different hydrolyzable groups (e.g., two or more esters comprising different hydrolyzable groups) are present in a molecule (e.g., an agent that provides a one-carbon unit). Use of different hydrolyzable groups can allow for selective hydrolysis of a particular ester. For example, one hydrolyzable group can be stable to acidic environments and the other can be stable to basic environments. In an alternative embodiment, one hydrolyzable group can be a hydrolyzable group cleaved by a particular enzyme, while the other is not cleaved by that enzyme.

In some embodiments, the hydrolysis of two or more hydrolyzable groups can occur simultaneously. Alternatively, the hydrolysis of the two or more hydrolyzable groups can be step-wise. Methods for the selection, introduction and subsequent removal of hydrolyzable groups are well known to those skilled in the art. (T. W. Greene and P. G. M. Wuts “Protective Groups in Organic Synthesis” John Wiley & Sons, Inc., New York 1999).

A prodrug can be derived from a polyol, natural sugar or unnatural sugar (e.g., glycerol, erythritol, xylitol, sorbitol, ribose, 2-deoxyribose, fructose, glucose, galactose, mannose, allose, altrose, gulose, idose, talose, xylose, maltitol, isomalt). Specific examples of prodrugs of formic acid derived from a polyol, natural sugar or unnatural sugar include, but are not limited to:

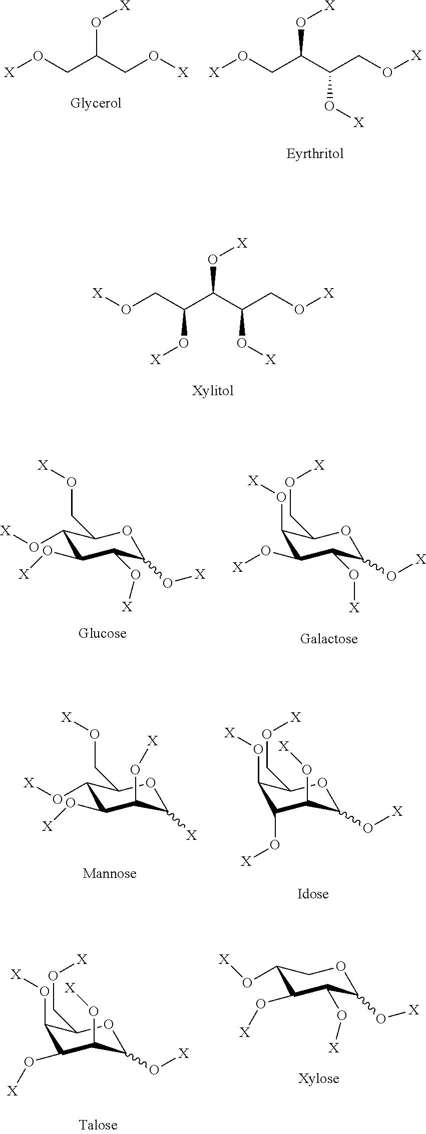

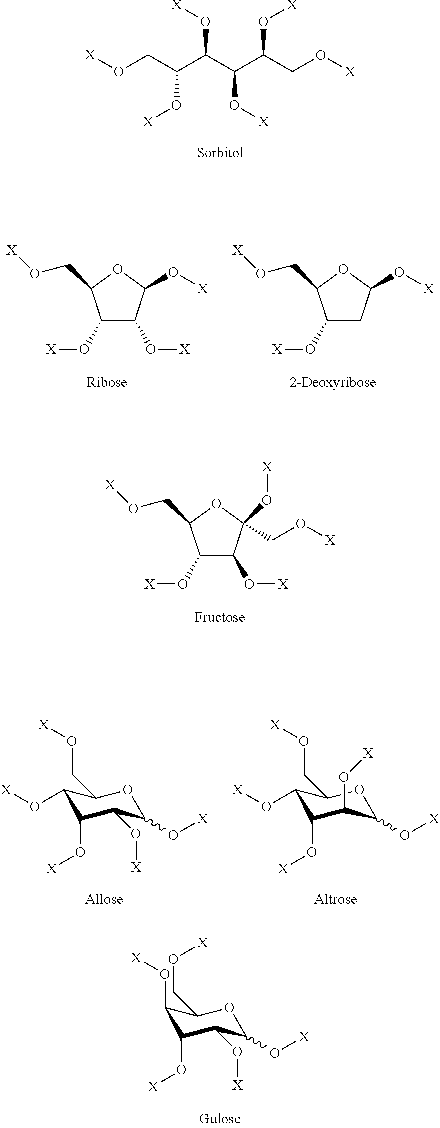

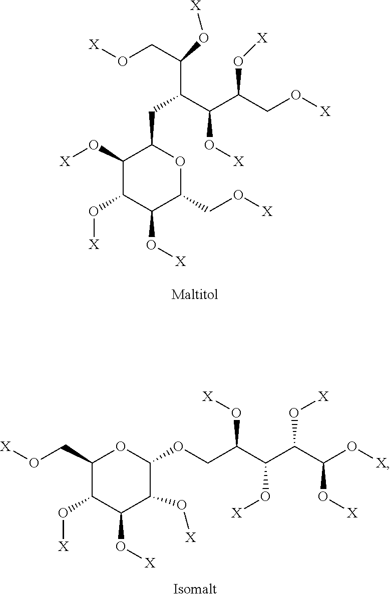

or a salt of any of the foregoing, wherein each X is independently hydrogen or formyl. Specific examples of prodrugs of formic acid comprising a (C₁-C₁₀)alkyl hydrolyzable group include, but are not limited to, methyl formate, ethyl formate, isopropyl formate and n-butyl formate. A specific example of a prodrug of formic acid derived from a polyethylene glycol is

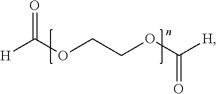

wherein n is an integer from 1 to about 100, for example, from 1 to about 50, from 1 to about 25, from 1 to about 10 or from 1 to about 5.

Prodrugs (e.g., ester prodrugs, such as ester prodrugs of formic acid) can also be derived from endogenous, naturally occurring, synthetic or approved food additives. Prodrugs (e.g., ester prodrugs, such as ester prodrugs of formic acid) can be absorbed through passive diffusion (as when the prodrug has a high degree of formylation) or through active transport, such as Na⁺/glucose transport (as when the prodrug has a relatively low degree of formylation).

The compounds described herein may be present in the form of salts (e.g., pharmaceutically acceptable salts). For use in medicines, the salts of the compounds described herein refer to non-toxic pharmaceutically acceptable salts. The pharmaceutically acceptable salts of the disclosed compounds include acid addition salts and base addition salts. The term “pharmaceutically acceptable salts” embraces salts commonly used to form alkali metal salts and to form addition salts of free acids or free bases. The nature of the salt is not critical, provided that it is pharmaceutically acceptable.

Suitable pharmaceutically acceptable acid addition salts of the disclosed compounds may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycolylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts of the disclosed compounds include, but are not limited to, metallic salts made from aluminum, calcium, lithium, magnesium, potassium, sodium and zinc or organic salts made from N,N′-dibenzylethylenediamine, chloroprocaine, choline, diethanolamine, ethylenediamine, N-methylglucamine, lysine, arginine and procaine. All of these salts may be prepared by conventional means from the corresponding compound represented by the disclosed compound by treating, for example, the disclosed compounds with the appropriate acid or base. Pharmaceutically acceptable basic/cationic salts also include the diethanolamine, ammonium, ethanolamine, piperazine and triethanolamine salts.

As used herein, the phrase “promoting an immune response” encompasses initiating, maintaining and/or enhancing an immune response. Examples of immune responses that can be promoted using the methods and compositions described herein include, but are not limited to, a T cell response, a macrophage response, an NK cell response, a dendritic cell response, a neutrophil response and a B cell response. In a particular embodiment, the immune response is a T cell response or an effector T cell response. In certain embodiments, “promoting an immune response” encompasses inhibiting or decreasing a Treg response. In a particular embodiment, the immune response is an immune response to a tumor or tumor antigen, also referred to herein as an “anti-tumor immune response”. An anti-tumor response can be directed to, for example, tumor control, (e.g., delaying and/or halting tumor growth and/or metastasis), tumor killing (e.g., causing the death of cancerous cells in a tumor), or both. In another embodiment, the immune response is an immune response to a vaccine.

Agents that are suitable for inhibiting (e.g., preventing, decreasing) the consumption of metabolic fuels (e.g., glucose) by tumor cells include, for example, agents that alter (e.g., inhibit) the activity (e.g., one or more enzymatic activities) of a metabolic enzyme or metabolic transporter. Alternatively, the agent can alter (e.g., decrease) the expression (e.g., transcription, mRNA processing, translation) of a metabolic enzyme or transporter gene or gene product (e.g., mRNA, protein).

Examples of metabolic enzymes include, but are not limited to indoleamine 2,3-dioxygenase (IDO), arginase, glutaminase, hexokinase, phosphoglucose isomerase, phosphofructokinase, fructose-1,6-bisphosphate aldolase, phosphofructokinase-2 (e.g., PFKFB3), triose phosphate isomerase, glyceraldehyde-3-phosphate dehydrogenase, phosphoglycerate kinase, phosphoglycerate mutase, enolase, pyruvate kinase and lactate dehydrogenase. Examples of metabolic transporters include, but are not limited to, glucose transporters and lactate transporters (e.g., MCT1 and MCT4).

In some embodiments, the agent is an inhibitor of glucose metabolism. Inhibitors of glucose metabolism include, for example, enzymes that inhibit glucose metabolism and agents that inhibit glucose transport. Examples of enzymes that inhibit glucose metabolism include, but are not limited to, fructose-1,6-bisphosphatase, phosphatase domain of fructose-2,6-bisphosphatase, a phosphofructokinase-2 isozyme with high phosphatase activity (e.g., PFKFB2), TIGAR, and PTEN.

In one embodiment, the inhibitor of glucose metabolism is an agent that inhibits a glucose transporter (e.g., GLUT1, GLUT2, GLUT3, GLUT4, and GLUT5). In a particular embodiment, the agent is an inhibitor of GLUT1. In another embodiment, the agent is an inhibitor of GLUT3.

In other embodiments, the inhibitor of glucose metabolism is an agent that inhibits a lactate transporter. Examples of lactate transporters include, monocarboxylate transport (MCT) proteins, among others.

In some embodiments, the agent acts specifically on tumor cells. For example, the agent inhibits metabolic fuel utilization by tumor cells without substantially affecting metabolic fuel utilization by immune cells in or surrounding the tumor, or elsewhere in the subject.

Suitable agents for inhibiting the consumption of metabolic fuels by tumor cells include, for example, small molecules, peptides, peptidomimetic compounds, antibodies, and nucleic acids, among others. Such agents can be naturally-occurring, synthetic or recombinant.

In an embodiment, the agent for inhibiting the consumption of metabolic fuels by tumor cells is a small molecule. Examples of small molecules include organic compounds, organometallic compounds, inorganic compounds, and salts of organic, organometallic and inorganic compounds. Atoms in a small molecule are typically linked together via covalent and/or ionic bonds. The arrangement of atoms in a small organic molecule may represent a chain (e.g. a carbon-carbon chain or a carbon-heteroatom chain), or may represent a ring containing carbon atoms, e.g. benzene or a polycyclic system, or a combination of carbon and heteroatoms, i.e., heterocycles such as a pyrimidine or quinazoline. Small molecule inhibitors generally have a molecular weight that is less than about 5,000 daltons. For example, such small molecules can be less than about 1000 daltons, less than about 750 daltons or even less than about 500 daltons. Small molecules and other non-peptidic metabolic enzyme inhibitors can be found in nature (e.g., identified, isolated, purified) and/or produced synthetically (e.g., by traditional organic synthesis, bio-mediated synthesis, or a combination thereof). See e.g. Ganesan, Drug Discov. Today 7(1): 47-55 (January 2002); Lou, Drug Discov. Today, 6(24): 1288-1294 (December 2001). Examples of naturally occurring small molecules include, but are not limited to, hormones, neurotransmitters, nucleotides, amino acids, sugars, lipids, and their derivatives.

Various small molecule inhibitors of metabolic fuel consumption are known in the art. In a particular embodiment, the small molecule is a GLUT1 inhibitor or a GLUT3 inhibitor. In certain embodiments, the small molecule inhibits GLUT3 to a greater extent than GLUT1.

In another embodiment, the agent for inhibiting the consumption of metabolic fuels by tumor cells is a nucleic acid. The term “nucleic acid” refers to a polymer having multiple nucleotide monomers. A nucleic acid can be single- or double-stranded, and can be DNA (e.g., cDNA or genomic DNA), RNA, or hybrid polymers (e.g., DNA/RNA). Nucleic acids can be chemically or biochemically modified and/or can contain non-natural or derivatized nucleotide bases. Nucleic acids can also include, for example, conformationally restricted nucleic acids (e.g., “locked nucleic acids” or “LNAs,” such as described in Nielsen et al., J. Biomol. Struct. Dyn. 17:175-91, 1999), morpholinos, glycol nucleic acids (GNA) and threose nucleic acids (TNA).

In a particular embodiment, the nucleic acid inhibits the expression (e.g., transcription, mRNA processing, translation) of a metabolic enzyme (e.g., hexokinase) or metabolic transporter (e.g., GLUT1) gene or gene product (e.g., mRNA, protein). Examples of nucleic acids that are suitable for inhibiting the expression of a metabolic enzyme or metabolic transporter include, but are not limited to, shRNAs, siRNAs, antisense nucleic acids (RNA or DNA), microRNAs, ribozymes and aptamers.

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence which is substantially identical to a nucleic acid sequence contained within the target gene product.

One or both strands of the siRNA can also comprise a 3′ overhang. As used herein, a “3′ overhang” refers to at least one unpaired nucleotide extending from the 3′-end of a duplexed RNA strand. Thus, in one embodiment, the siRNA comprises at least one 3′ overhang of from 1 to about 6 nucleotides (which includes ribonucleotides or deoxyribonucleotides) in length, preferably from 1 to about 5 nucleotides in length, more preferably from 1 to about 4 nucleotides in length, and particularly preferably from about 2 to about 4 nucleotides in length. In a preferred embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated miR gene products. Exemplary methods for producing and testing dsRNA or siRNA molecules are described in U.S. published patent application 2002/0173478 to Gewirtz and in U.S. published patent application 2004/0018176 to Reich et al., the entire disclosures of which are herein incorporated by reference.

Antisense nucleic acids suitable for use in the present methods are typically single-stranded nucleic acids (e.g., RNA, DNA, LNA, RNA-DNA chimeras, PNA) that comprise a nucleic acid sequence that is complementary to a contiguous nucleic acid sequence in a target gene product. In some embodiments, antisense nucleic acids can contain one or more chemical modifications (e.g., cholesterol moieties, duplex intercalators such as acridine, or nuclease-resistant groups) to the nucleic acid backbone, the sugar, the base moieties (or their equivalent), or a combination thereof.

In certain embodiments, the agent is delivered by administering to the subject a nucleic acid that encodes the agent (e.g., by localized administration to the tumor). Typically, the nucleic acid that encodes the agent will be included in a gene delivery vector that is suitable for gene therapy methods.

The terms “vector”, “vector construct” and “expression vector” mean the vehicle by which a DNA or RNA sequence (e.g. a foreign gene) can be introduced into a host cell, so as to transform the host and promote expression (e.g. transcription and translation) of the introduced sequence. Vectors typically comprise the DNA of a transmissible agent, into which foreign DNA encoding a protein is inserted by restriction enzyme technology. A common type of vector is a “plasmid”, which generally is a self-contained molecule of double-stranded DNA that can readily accept additional (foreign) DNA and which can readily be introduced into a suitable host cell. A large number of vectors, including plasmid and fungal vectors, have been described for replication and/or expression in a variety of eukaryotic and prokaryotic hosts.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

Gene delivery vectors generally include a transgene (e.g., nucleic acid encoding an agent, such as an shRNA or enzyme that inhibits glucose metabolism) operably linked to a promoter and other nucleic acid elements required for expression of the transgene in tumor cells. Suitable promoters for gene expression and delivery constructs are known in the art and include, for example, the U6 or H1 RNA pol III promoter sequences, or cytomegalovirus (CMV) promoters. The selection of a suitable promoter is within the skill in the art. The recombinant plasmids of the invention can also comprise inducible, or regulatable, promoters for expression of an inhibitor compound in cells.

Various gene delivery vehicles for gene therapy are known in the art and include both viral and non-viral (e.g., naked DNA, plasmid) vectors. Viral vectors commonly used in gene therapy in mammals, including humans, are known to those skilled in the art. Such viral vectors include, e.g., vector derived from the herpes virus, baculovirus vector, lentiviral vector, retroviral vector, adenoviral vector and adeno-associated viral vector (AAV). The viral vector can be replicating or non-replicating

Non-viral vectors include naked DNA and plasmids, among others. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art.

In certain methods of the invention, the vector comprises a transgene operably linked to a promoter. The transgene encodes a biologically active molecule, expression of which in the CNS results in at least partial correction of storage pathology and/or stabilization of disease progression.

To facilitate the introduction of the gene delivery vector into tumor cells, the vector can be combined with different chemical means such as colloidal dispersion systems (macromolecular complex, nanocapsules, microspheres, beads) or lipid-based systems (oil-in-water emulsions, micelles, liposomes).

Agents that inhibit the consumption of metabolic fuels (e.g., glucose) by tumor cells, and/or cells (e.g., immune cells) that express an exogenous enzyme that catalyzes the oxidation NADH to NAD⁺, can be administered to a subject in need thereof by a variety of routes of administration including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the agent and the particular cancer to be treated. Methods for administering a population of immune cells (e.g., an ex vivo population), such as CAR-T cells, to a subject are well-known in the art.

Agents that provide one-carbon units can be administered to a subject in need thereof by a variety of routes of administration including, for example, oral (e.g., dietary), topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the agent and the particular cancer to be treated. In some embodiments, an agent that provides a one-carbon unit is administered to a subject orally (e.g., in the form of a nutritional supplement).

Administration can be local or systemic as indicated. The chosen mode of administration can vary depending on the particular agent selected. For example, in gene therapy-based methods, a nucleic acid encoding an agent that inhibits consumption of metabolic fuels by tumor cells is administered locally, such as intratumorally. Techniques for intratumoral delivery of therapeutic agents are known in the art and include, for example, intratumoral injection and intratumoral infusion. The actual dose of a therapeutic agent and treatment regimen can be determined by a skilled physician, taking into account the nature of the condition being treated, and patient characteristics.

As used herein, “subject” refers to a mammal (e.g., human, such as an aged human, non-human primate, cow, sheep, goat, horse, dog, cat, rabbit, guinea pig, rat, mouse). In a particular embodiment, the subject is a human. As used herein, “aged human” means a human who is greater than about 40, about 50, about 55, about 60, about 65, about 70, about 75, about 80, about 85, or about 90 years old. A “subject in need thereof” refers to a subject (e.g., patient) who has, or is at risk for developing, a disease or condition that can be treated (e.g., improved, ameliorated, prevented) by an immunotherapy.

As used herein, the terms “treat,” “treating,” or “treatment,” mean to counteract a medical condition (e.g., a condition related to cancer) to the extent that the medical condition is improved according to a clinically-acceptable standard (e.g., reduction in tumor formation, size, growth or metastasis).

In an embodiment, the subject in need thereof has cancer. The cancer can be a solid tumor, a leukemia, a lymphoma or a myeloma. In particular embodiments, the subject in need thereof has a solid tumor, such as a breast tumor, a colon tumor, a lung tumor, a pancreatic tumor, a prostate tumor, a bone tumor, a skin tumor (e.g., melanoma, squamous cell carcinoma), a brain tumor, a head and neck tumor, a lymphoid tumor, or a liver tumor. In particular embodiments, the subject in need thereof has a solid tumor, such as a breast tumor, an ovarian tumor, a colon tumor, a lung tumor, a pancreatic tumor, a prostate tumor, a bone tumor, a skin tumor (e.g., melanoma, squamous cell carcinoma), a brain tumor, a head and neck tumor, a lymphoid tumor, or a liver tumor. In certain embodiments, the subject has a solid tumor having one or more features selected from poor perfusion, a low NAD⁺/NADH ratio, a low oxygen (O₂) level, and a high lactate level. In some embodiments, the subject has a metastatic cancer, such as a metastatic lung cancer. In some embodiments, the subject has lung cancer (e.g., a lung tumor), such as non-small cell lung cancer (NSCLC). Lung cancer can be smoking-induced lung cancer or non-smoking-induced lung cancer. In some embodiments, the lung cancer carries a high mutation burden or a high rate of somatic mutation, such as that observed in bladder cancer, melanoma, squamous lung cancer and lung adenocarcinoma. Although not wishing to be bound by any particular theory, it is generally believed that the degree of somatic mutation or neo-epitope burden generally correlates with positive response to immunotherapy.

Exemplary cancers include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Mantle Cell Lymphoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macroglobulinemia; and Wilms' Tumor.

One embodiment is a method of treating cancer (e.g., a tumor, such as a solid tumor) in a subject in need thereof, comprising administering to the subject (e.g., a human, such as an aged human) an effective amount of an agent that provides a one-carbon unit and an agent that promotes an anti-tumor response. In a particular embodiment, the cancer is lung cancer (e.g., NSCLC). In some embodiments, the lung cancer is smoking-induced lung cancer. In some embodiments, the lung cancer is non-smoking-induced lung cancer. In some embodiments, the lung cancer is lung cancer with a high mutation burden. In some embodiments, the cancer is mesothelioma. In some embodiments, the cancer is a metastatic cancer, such as a metastatic lung cancer.

Agents that provide a one-carbon unit and agents that promote an anti-tumor response suitable for use in methods of treating cancer include those described herein and combinations thereof. In some embodiments, the agent that provides a one-carbon unit is formic acid, a prodrug thereof or a pharmaceutically acceptable salt thereof. In some embodiments, the agent that provides a one-carbon unit is folic acid, 5-methyl-THF, 5-formyl-THF, a prodrug of any of the foregoing or a pharmaceutically acceptable salt of any of the foregoing. In some embodiments, at least two agents that provides a one-carbon unit are administered. In some embodiments, at least two agents that provide a one-carbon unit are administered, wherein the at least two agents that provide a one-carbon unit include formic acid, a prodrug thereof or a salt of either of the foregoing, and glycine, a prodrug thereof or a salt of either of the foregoing. In some embodiments, the agent that promotes an anti-tumor response is an antibody, a vaccine or a population of immune cells. In some embodiments, the agent that promotes an anti-tumor response is an agent (e.g., an antibody) that inhibits PD-1.

In certain embodiments, an effective amount of an agent that inhibits the consumption of metabolic fuels (e.g., glucose) by tumor cells is administered to a subject in need thereof. In certain embodiments, an effective amount of an agent that provides a one-carbon unit is administered to a subject in need thereof. As defined herein, an “effective amount” refers to an amount of agent that, when administered to a subject, is sufficient to achieve a desired therapeutic effect in the subject under the conditions of administration, such as an amount sufficient to promote (e.g., initiate, maintain and/or enhance) an immune response (e.g., a T cell response) to a tumor in the subject. Various methods of measuring immune responses, including T cell responses, are known in the art. For example, promotion of a T cell response can be assessed by detecting increased levels of activated T cells in the tumor and/or the tumor microenvironment following administration of the agent or nucleic acid encoding the agent. T cell subsets can be assessed by immunohistochemistry or FACS sorting.

The therapeutic effectiveness of an agent that inhibits the consumption of metabolic fuels (e.g., glucose) by tumor cells can be determined by any suitable method known to those of skill in the art (e.g., in situ immunohistochemistry, imaging (ultrasound, CT scan, MRI, NMR), ³H-thymidine incorporation) using any suitable standard (e.g., inhibition of tumor formation, tumor growth (proliferation, size), tumor vascularization, tumor progression (invasion, metastasis) and/or chemoresistance).

The therapeutic effectiveness of an agent that provides a one-carbon unit can be determined by any suitable method known to those of skill in the art (e.g., in situ immunohistochemistry, imaging (ultrasound, CT scan, MRI, NMR), ³H-thymidine incorporation) using any suitable standard (e.g., inhibition of tumor formation, tumor growth (proliferation, size), tumor vascularization, tumor progression (invasion, metastasis) and/or chemoresistance).

An effective amount of the agent(s) to be administered can be determined by a clinician of ordinary skill using the guidance provided herein and other methods known in the art, and is dependent on several factors including, for example, the particular agent(s) chosen, the subject's age, sensitivity, tolerance to drugs and overall well-being. For example, suitable dosages for a small molecule can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Suitable dosages for antibodies can be from about 0.01 mg/kg to about 300 mg/kg body weight per treatment and preferably from about 0.01 mg/kg to about 100 mg/kg, from about 0.01 mg/kg to about 10 mg/kg, from about 1 mg/kg to about 10 mg/kg body weight per treatment. Where the agent is a polypeptide (linear, cyclic, mimetic), the preferred dosage will typically result in a plasma concentration of the peptide from about 0.1 μg/mL to about 200 μg/mL. Determining the dosage for a particular agent, patient and cancer is well within the abilities of one of skill in the art. Preferably, the dosage does not cause or produces minimal adverse side effects (e.g., immunogenic response, nausea, dizziness, gastric upset, hyperviscosity syndromes, congestive heart failure, stroke, pulmonary edema).

An agent that inhibits the consumption of metabolic fuels (e.g., glucose) by tumor cells can be administered in a single dose or as multiple doses, for example, in an order and on a schedule suitable to achieve a desired therapeutic effect (e.g., promotion of an anti-tumor immune response). Suitable dosages and regimens of administration can be determined by a clinician of ordinary skill. With respect to the administration of an agent in combination with one or more other therapies or treatments (adjuvant, targeted, cancer treatment-associated, and the like), the agent is typically administered as a single dose (by, e.g., injection, infusion, orally), followed by repeated doses at particular intervals (e.g., one or more hours) if desired or indicated.

An agent that provides a one-carbon unit can be administered in a single dose or as multiple doses, for example, in an order and on a schedule suitable to achieve a desired therapeutic effect (e.g., promotion of an anti-tumor immune response). Suitable dosages and regimens of administration can be determined by a clinician of ordinary skill. With respect to the administration of an agent in combination with one or more other therapies or treatments (adjuvant, targeted, cancer treatment-associated, and the like), the agent is typically administered as a single dose (by, e.g., injection, infusion, orally), followed by repeated doses at particular intervals (e.g., one or more hours) if desired or indicated.

An agent that inhibits the consumption of metabolic fuels (e.g., glucose) by tumor cells can be administered to the subject in need thereof as a primary therapy (e.g., as the principal therapeutic agent in a therapy or treatment regimen); as an adjunct therapy (e.g., as a therapeutic agent used together with another therapeutic agent in a therapy or treatment regime, wherein the combination of therapeutic agents provides the desired treatment; “adjunct therapy” is also referred to as “adjunctive therapy”); in combination with an adjunct therapy; as an adjuvant therapy (e.g., as a therapeutic agent that is given to the subject in need thereof after the principal therapeutic agent in a therapy or treatment regimen has been given); or in combination with an adjuvant therapy. Adjuvant therapies include, for example, chemotherapy (e.g., paclitaxel, doxorubicin, tamoxifen, cisplatin, mitomycin, 5-fluorouracil, sorafenib, octreotide, dacarbazine (DTIC), cis-platinum, cimetidine, cyclophosphamide), radiation therapy (e.g., proton beam therapy), hormone therapy (e.g., anti-estrogen therapy, androgen deprivation therapy (ADT), luteinizing hormone-releasing hormone (LH-RH) agonists, aromatase inhibitors (AIs, such as anastrozole, exemestane, letrozole), estrogen receptor modulators (e.g., tamoxifen, raloxifene, toremifene)), or biological therapy. Numerous other therapies can also be administered during a cancer treatment regime to mitigate the effects of the disease and/or side effects of the cancer treatment including therapies to manage pain (narcotics, acupuncture), gastric discomfort (antacids), dizziness (anti-vertigo medications), nausea (anti-nausea medications), infection (e.g., medications to increase red/white blood cell counts) and the like, all of which are readily appreciated by the person skilled in the art.

An agent that provides a one-carbon unit can be administered to the subject in need thereof as a primary therapy (e.g., as the principal therapeutic agent in a therapy or treatment regimen); as an adjunct therapy (e.g., as a therapeutic agent used together with another therapeutic agent in a therapy or treatment regime, wherein the combination of therapeutic agents provides the desired treatment; “adjunct therapy” is also referred to as “adjunctive therapy”); in combination with an adjunct therapy; as an adjuvant therapy (e.g., as a therapeutic agent that is given to the subject in need thereof after the principal therapeutic agent in a therapy or treatment regimen has been given); or in combination with an adjuvant therapy. Adjuvant therapies include, for example, chemotherapy (e.g., paclitaxel, doxorubicin, tamoxifen, cisplatin, mitomycin, 5-fluorouracil, sorafenib, octreotide, dacarbazine (DTIC), cis-platinum, cimetidine, cyclophosphamide), radiation therapy (e.g., proton beam therapy), hormone therapy (e.g., anti-estrogen therapy, androgen deprivation therapy (ADT), luteinizing hormone-releasing hormone (LH-RH) agonists, aromatase inhibitors (AIs, such as anastrozole, exemestane, letrozole), estrogen receptor modulators (e.g., tamoxifen, raloxifene, toremifene)), or biological therapy. Numerous other therapies can also be administered during a cancer treatment regime to mitigate the effects of the disease and/or side effects of the cancer treatment including therapies to manage pain (narcotics, acupuncture), gastric discomfort (antacids), dizziness (anti-vertigo medications), nausea (anti-nausea medications), infection (e.g., medications to increase red/white blood cell counts) and the like, all of which are readily appreciated by the person skilled in the art.

In some embodiments, the method comprises administering an effective amount of an agent that inhibits the consumption of metabolic fuels (e.g., glucose) by tumor cells in combination with one or more additional therapeutic agents (e.g., additional agents that inhibit consumption of metabolic fuels by tumor cells, agents that promote an anti-tumor response) or therapies (e.g., chemotherapy, radiation and/or the surgical removal of a tumor(s)).

Examples of chemotherapeutic agents include, for example, antimetabolites (e.g., folic acid, purine, pyrimidine derivatives) and alkylating agents (e.g., nitrogen mustards, nitrosoureas, platinum, alkyl sulfonates, hydrazines, triazenes, aziridines, spindle poison, cytotoxic agents, topoisomerase inhibitors), Aclarubicin, Actinomycin, Alitretinon, Altretamine, Aminopterin, Aminolevulinic acid, Amrubicin, Amsacrine, Anagrelide, Arsenic trioxide, Asparaginase, Atrasentan, Belotecan, Bexarotene, Bendamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat and Zorubicin.

In some embodiments, the method comprises administering an effective amount of an agent that provides a one-carbon unit in combination with one or more additional therapeutic agents (e.g., additional agents that provide a one-carbon unit, agents that promote an anti-tumor response) or therapies (e.g., chemotherapy, radiation and/or the surgical removal of a tumor(s)).

When administered in a combination therapy, the agent (e.g., agent that inhibits consumption of metabolic fuels, agent that provides a one-carbon unit) can be administered before, after or concurrently with the other therapy (e.g., administration of a chemotherapeutic agent, such a paclitaxel or doxorubicin). When co-administered simultaneously (e.g., concurrently), the agent and other therapy can be in separate formulations or the same formulation. Alternatively, the agent and other therapy can be administered sequentially, as separate compositions, within an appropriate time frame (e.g., a cancer treatment session/interval such as 1.5 to 5 hours) as determined by a skilled clinician (e.g., a time sufficient to allow an overlap of the pharmaceutical effects of the therapies).

In certain embodiments, an agent (e.g., an effective amount of an agent) that inhibits the consumption of metabolic fuels (e.g., glucose) by tumor cells (or nucleic acid encoding an agent that inhibits consumption of metabolic fuels by tumor cells) is administered to a subject in combination with one or more additional agents (e.g., 1, 2, 3, 4, etc. additional agents, such as an effective amount of 1, 2, 3, 4, etc. additional agents), or nucleic acids encoding one or more additional agents, that are useful for inhibiting consumption of metabolic fuels by tumor cells. For example, the additional agents can target downstream steps in glycolysis (e.g., by targeting hexokinase), multiple isozymes at the same step (e.g. GLUT1+GLUT3) and/or multiple enzymes at different steps (e.g. GLUT1+HK2+MCT4).

In certain embodiments, an agent (e.g., an effective amount of an agent) that provides a one-carbon unit is administered to a subject in combination with one or more additional agents (e.g., 1, 2, 3, 4, etc. additional agents, such as an effective amount of 1, 2, 3, 4, etc. additional agents), or nucleic acids encoding one or more additional agents, that are useful for inhibiting consumption of metabolic fuels by tumor cells. For example, the additional agents can target downstream steps in glycolysis (e.g., by targeting hexokinase), multiple isozymes at the same step (e.g. GLUT1+GLUT3) and/or multiple enzymes at different steps (e.g. GLUT1+HK2+MCT4).

In some embodiments, an agent (e.g., an effective amount of an agent) that inhibits the consumption of metabolic fuels (e.g., glucose) by tumor cells (or nucleic acid encoding an agent that inhibits consumption of metabolic fuels by tumor cells) is administered to a subject in combination with one or more additional agents (e.g., 1, 2, 3, 4, etc. additional agents, such as an effective amount of 1, 2, 3, 4, etc. additional agents), or nucleic acids encoding one or more additional agents, that are useful for promoting anti-tumor responses (e.g., agents that inhibit PD-1 or PD-L1). In some embodiments, an agent (e.g., an effective amount of an agent) that provides a one-carbon unit is administered to a subject in combination with one or more additional agents (e.g., 1, 2, 3, 4, etc. additional agents, such as an effective amount of 1, 2, 3, 4, etc. additional agents), or nucleic acids encoding one or more additional agents, that are useful for promoting anti-tumor responses (e.g., agents that inhibit PD-1 or PD-L1).

As used herein, “agent useful for promoting an anti-tumor response” and “agent that promotes an anti-tumor response” are cancer immunotherapy agents. Cancer immunotherapy refers to a diverse set of therapeutic strategies designed to induce a subject's own immune system to fight a tumor. Cancer immunotherapy agents include antibodies that inhibit proteins expressed by cancer cells, vaccines and immune cell (e.g., T-cell) infusions. Antibody agents useful for promoting anti-tumor responses include anti-CTLA-4 antibodies (e.g., ipilimumab, tremelimumab), anti-PD-1 antibodies (e.g., nivolumab, pembrolizumab), anti-PD-L1 antibodies (e.g., avelumab), anti-PD-L2 antibodies, anti-TIM-3 antibodies, anti-LAG-3 antibodies, anti-OX40 antibodies and anti-GITR antibodies. In some embodiments, the agent that promotes an anti-tumor response is an anti-PD-1 antibody, an anti-PD-L1 antibody or an anti CTLA-4 antibody or, in more specific embodiments, an anti-PD-1 antibody. In the context of agents useful for promoting an anti-tumor response and agents that promote an anti-tumor response, the phrases “agent useful for promoting an anti-tumor response,” “agent that promotes an anti-tumor response” and “agent that promotes an anti-tumor immune response” can be used interchangeably.

Agents that provide a one-carbon unit and agents that promote an anti-tumor response suitable for use in methods of promoting an immune response to a tumor include those described herein and combinations thereof. In some embodiments, the agent that provides a one-carbon unit is formic acid, a prodrug thereof or a pharmaceutically acceptable salt thereof. In some embodiments, the agent that provides a one-carbon unit is folic acid, 5-methyl-Tf, 5-formyl-THF, a prodrug of any of the foregoing or a pharmaceutically acceptable salt of any of the foregoing. In some embodiments, at least two agents that provide a one-carbon unit are administered. In some embodiments, at least two agents that provide a one-carbon unit are administered, wherein the at least two agents that provide a one-carbon unit include formic acid, a prodrug thereof or a salt of either of the foregoing, and glycine, a prodrug thereof or a salt of either of the foregoing. In some embodiments, the agent that promotes an anti-tumor response is an antibody, a vaccine or a population of immune cells. In some embodiments, the agent that promotes an anti-tumor response is an agent (e.g., an antibody) that inhibits PD-1. In a particular embodiment, an effective amount of an agent that provides a one-carbon unit (e.g., a source of a one-carbon unit, such as any of the sources of one-carbon units described herein) is administered to a subject in combination with an effective amount of one or more agents that promote an anti-tumor response (e.g., an antibody, vaccine or population of immune cells, such as an antibody that inhibits PD-1). In a more particular embodiment, the agent that provides a one-carbon unit is formic acid or a prodrug thereof, or a pharmaceutically acceptable salt of either of the foregoing, and the agent that promotes an anti-tumor response is an agent that inhibits PD-1 (e.g., an antibody that inhibits PD-1).

Aging results in numerous biological changes, including a disadvantageous propensity for increased overall inflammation and/or decrease in effective antigen-specific immune responses. This includes less effective immune responses to bacteria, viruses, parasites, and cancer. It further includes less effective responses to vaccination. One embodiment of the present invention is a method of treating immune dysfunction in a subject in need thereof, including an aged human (e.g., a human greater than about 40, about 50, about 55, about 60, about 65, about 70, about 75, about 80, about 85, or about 90 years old), comprising administering to the subject an effective amount of an agent that provides a one-carbon unit, such as formic acid or a prodrug thereof, or a pharmaceutically acceptable salt of either of the foregoing. In certain embodiments, the method further comprises administering an effective amount of an agent that promotes an immune (e.g., anti-tumor) response, such as a vaccine.

Examples of agents that promote an immune response include vaccines (e.g., live whole virus vaccines, killed whole virus vaccines, subunit vaccines, recombinant virus vaccines, anti-idiotype antibodies, DNA vaccines) and agents that promote an anti-tumor response, including those described herein.

Administration of the agent that promotes an immune response can occur before, after or contemporaneously with administration of the agent that provides a one-carbon unit. Without wishing to be bound by any particular theory, it is believed that the combination of an agent that provides a one-carbon unit and an agent that promotes an immune response, such as a vaccine, will enhance the effectiveness of the vaccine. In certain embodiments, administration of an agent that provides a one-carbon unit remediates an age-induced immune dysfunction, including a defect in production of relevant immune cell subsets, cytokines, and/or antibodies.

Agents that provide a one-carbon unit and agents that promote an immune response suitable for use in methods of treating immune dysfunction include those described herein and combinations thereof. In some embodiments, the agent that provides a one-carbon unit is formic acid, a prodrug thereof or a pharmaceutically acceptable salt thereof. In some embodiments, the agent that provides a one-carbon unit is folic acid, 5-methyl-THF, 5-formyl-THF, a prodrug of any of the foregoing or a pharmaceutically acceptable salt of any of the foregoing. In some embodiments, at least two agents that provide a one-carbon unit are administered. In some embodiments, at least two agents that provide a one-carbon unit are administered, wherein the at least two agents that provide a one-carbon unit include formic acid, a prodrug thereof or a salt of either of the foregoing, and glycine, a prodrug thereof or a salt of either of the foregoing. In some embodiments, the agent that promotes an immune response is a vaccine. In some embodiments, the agent the promotes an immune response is an agent that promotes an anti-tumor response (e.g., an antibody, vaccine or population of immune cells; an agent that inhibits PD-1, such as an antibody that inhibits PD-1).

In some embodiments, an effective amount of an agent that inhibits the consumption of metabolic fuels (e.g., glucose) by tumor cells (or nucleic acid encoding an agent that inhibits consumption of metabolic fuels by tumor cells) is administered to a subject in combination with an effective amount of one or more additional agents (e.g., 1, 2, 3, 4, etc. additional agents), or nucleic acids encoding one or more additional agents, that are useful for decreasing or depleting suppressor T cells.

In some embodiments, an effective amount of an agent that provides a one-carbon unit is administered to a subject in combination with an effective amount of one or more additional agents (e.g., 1, 2, 3, 4, etc. additional agents), or nucleic acids encoding one or more additional agents, that are useful for decreasing or depleting suppressor T cells.

Compositions Comprising Populations of Immune Cells: Compositions Comprising Agents, or Nucleic Acids Encoding Agents, that Inhibit the Consumption of Metabolic Fuels

In additional embodiments, the present invention provides compositions comprising a population (e.g., ex vivo population) of immune cells expressing an exogenous enzyme that catalyzes the oxidation of nicotinamide adenine dinucleotide, reduced form (NADH) to nicotinamide adenine dinucleotide, oxidized form (NAD+). In a particular embodiment, the exogenous enzyme is an NADH oxidase described herein (e.g., an NADH oxidase from Lactobacillus brevis, a variant of a naturally occurring NADH oxidase that has been engineered for reduced immunogenicity in a human subject). In an embodiment, the NADH oxidase is coupled to a lactate dehydrogenase enzyme.

In an embodiment, the immune cells in the population include T cells (e.g., human T cells). The T cells can be cultured or uncultured. Methods of obtaining and/or preparing populations of T cells are known in the art.

In a particular embodiment, the immune cells are chimeric antigen receptor T cells (CAR-T cells). In a further embodiment, the CAR-T cells recognize an antigen on tumor cells, such as an antigen described herein. Suitable methods of obtaining and/or preparing populations of CAR-T cells are known in the art.

In some embodiments, the population (e.g., ex vivo population) of immune cells is in a culture medium. In further embodiments, the culture medium comprises an agent that provides a one-carbon unit (e.g., formic acid, a prodrug thereof or a salt of either of the foregoing; formic acid, a prodrug thereof or a salt of either of the foregoing and glycine, a prodrug thereof or a salt of either of the foregoing).

In certain embodiments, the immune cells in the population comprise a nucleic acid molecule (e.g., plasmid), or nucleic acid sequence insertion in the immune cell genome, that encodes an exogenous enzyme (e.g., an NADH oxidase) that catalyzes the oxidation of NADH to NAD⁺. Methods of introducing nucleic acid molecules into cells (e.g., immune cells) are well-known in the art and include the methods and techniques described herein (e.g., transfection). Methods for modulating the immune cell genome are also well-known in the art, including via use of CRISPR-Cas9. In an embodiment, the nucleic acid molecule that encodes an exogenous enzyme that catalyzes the oxidation of NADH to nicotinamide adenine dinucleotide NAD⁺ (e.g., an NADH oxidase) is a DNA expression vector (e.g., a plasmid). The DNA expression vector can be a viral vector, such as a lentiviral vector, or a non-viral vector.

In further embodiments, the invention provides compositions comprising agents, or nucleic acids encoding agents, that inhibit the consumption of metabolic fuels by tumor cells. The agent or nucleic acid can be administered as a neutral compound or as a salt or ester. Pharmaceutically acceptable salts include those described herein and those formed with free amino groups such as those derived from hydrochloric, phosphoric, acetic, oxalic or tartaric acids, and those formed with free carboxyl groups such as those derived from sodium, potassium, ammonium, calcium, ferric hydroxides, isopropylamine, triethylamine, 2-ethylamino ethanol, histidine, procaine, etc. Salts of compounds containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium or potassium.

In certain embodiments, the composition comprises a nucleic acid encoding an inhibitor of metabolic fuel consumption, and a pharmaceutically-acceptable carrier or excipient. In a particular embodiment, the composition comprises a nucleic acid expression construct encoding an inhibitor of glucose metabolism, and a pharmaceutically-acceptable carrier or excipient. In one embodiment, the inhibitor of glucose metabolism is an inhibitor of a glucose transporter (e.g., GLUT1, GLUT2, GLUT3, GLUT4, and GLUT5). In one embodiment, the composition comprises a nucleic acid expression construct encoding an inhibitor GLUT1, and a pharmaceutically-acceptable carrier or excipient

In some embodiments, the compositions of the invention comprise one or more pharmaceutically acceptable carriers or excipients. Suitable pharmaceutical carriers typically will contain inert ingredients that do not interact with the agent or nucleic acid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate, solutions appropriate for supporting the health of immune cells (e.g., solutions containing glucose, amino acids, growth factors, and/or other nutrients or immune stimulators), and the like. Formulations can also include small amounts of substances that enhance the effectiveness of the active ingredient (e.g., emulsifying agents, solubilizing agents, pH buffering agents, wetting agents). Methods of encapsulation compositions (such as in a coating of hard gelatin or cyclodextran) are known in the art. For inhalation, the agent can be solubilized and loaded into a suitable dispenser for administration (e.g., an atomizer or nebulizer or pressurized aerosol dispenser).

In some embodiments, the compositions of the invention include one or more other therapeutic agents (e.g., a chemotherapeutic agent, for example, paclitaxel, doxorubicin, 5-fluorouracil, tamoxifen, octreotide, and/or immunomodulatory compounds (e.g., antibodies against targets such as PD-1, PD-L1, or CTLA-4). In some embodiments, the compositions of the invention include (e.g., an effective amount of) at least one (e.g., 1, 2, 3, 4) agent that provides a one carbon unit (e.g., serine, glycine, histidine, tryptophan, formic acid, folic acid, 5-methyl-tetrahydrofolate; 5-formyl-THF, monomethylglycine, dimethylglycine, glycine betaine, choline and glucose, a prodrug (e.g., an ester prodrug, an amide prodrug) of any of the foregoing or a salt (e.g., a pharmaceutically acceptable salt) of any of the foregoing). In a particular embodiment, the composition includes two agents that provide a one carbon unit (e.g., formic acid and glycine, or a prodrug or pharmaceutically acceptable salt of either of the foregoing).

Standard pharmaceutical formulation techniques can be employed, such as those described in Remington's Pharmaceutical Sciences, Mack Publishing Company, Easton, PA.

### Exemplification

Example 1. Three groups of female BALB/c mice with established subcutaneous CT26 tumors (n=10/group, group mean tumor: 97 mm³) received formate (20 mg/mL) in the drinking water and intraperitoneal (i.p.) anti-PD-1 treatment (5 mg/kg, twice a week for two weeks), alone and in combination. An untreated group served as the control group for efficacy analysis. One group received the combination of anti-PD-1 (i.p., 5 mg/kg, twice a week for two weeks) and anti-CTLA-4 (i.p., 5 mg/kg on day 1, 2.5 mg/kg on days 4 and 7) antibodies as a positive control. The study endpoint was a tumor volume of 2000 mm³ or 45 days, whichever came first. The study was terminated on day 32 when all tumors in formate treatment groups reached 2000 mm³. Tumor measurements were taken twice weekly and animals exited the study upon reaching the tumor volume endpoint. Overall efficacy was determined from percent tumor growth delay (% TGD), the percent increase in the median time to endpoint (TTE) for a treatment group compared to the control group. Animals were also monitored for partial regression (PR) and complete regression (CR) responses. Treatment tolerability was assessed by frequent observation for clinical signs of treatment-related (TR) side effects and by monitoring body weight (BW).

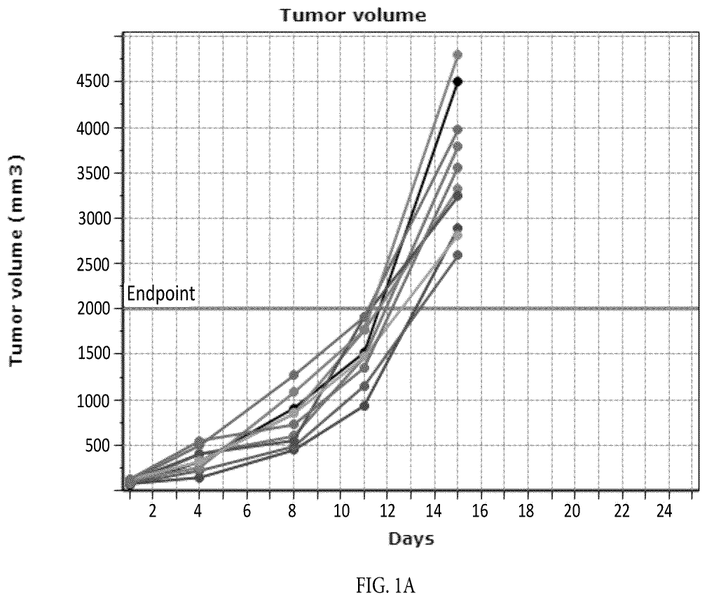

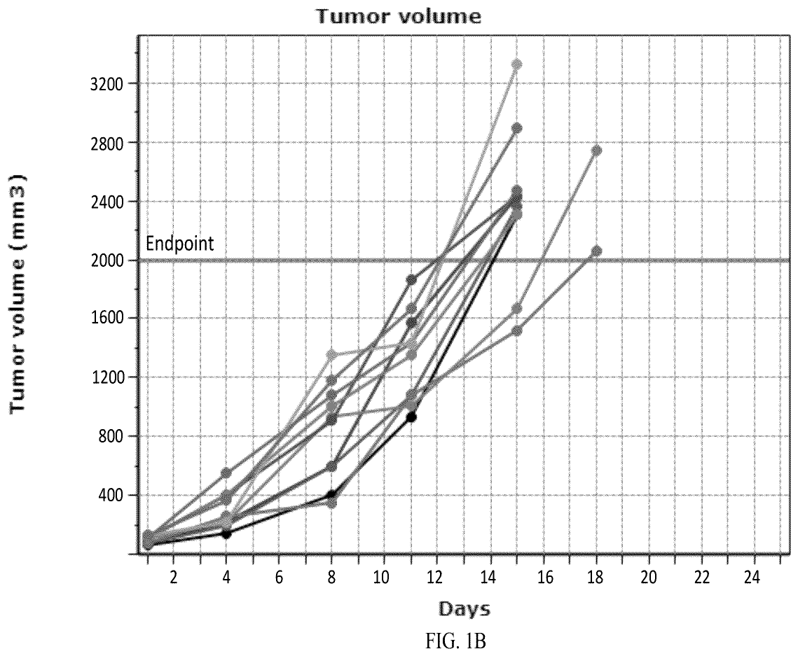

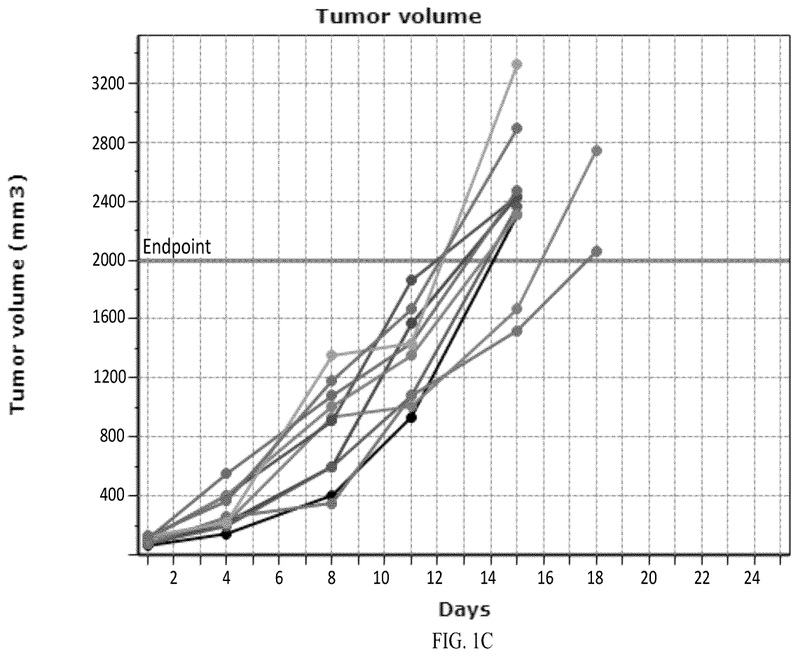

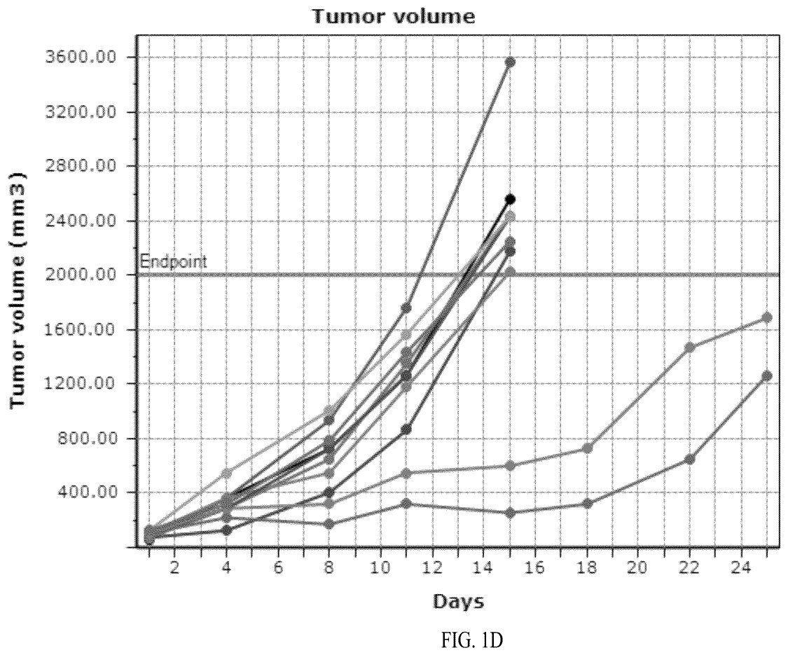

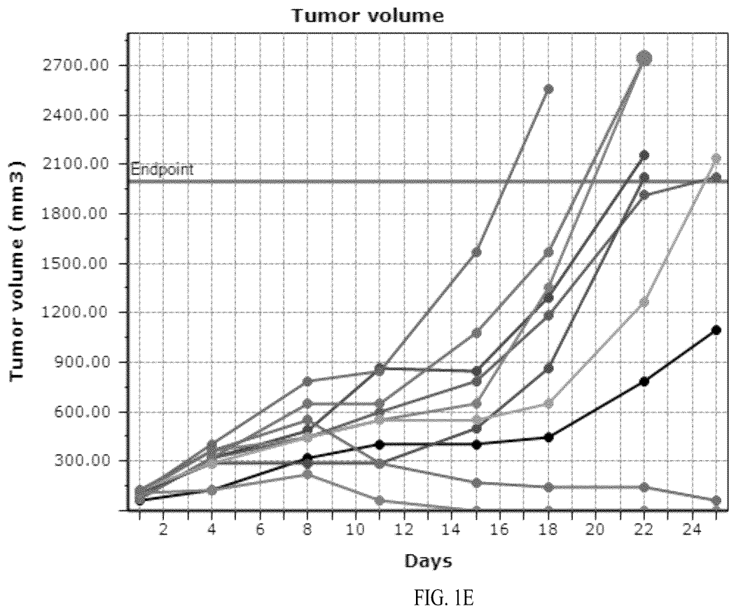

FIGS. 1A-1E show the individual tumor growth trajectories (as measured biweekly per the study protocol).

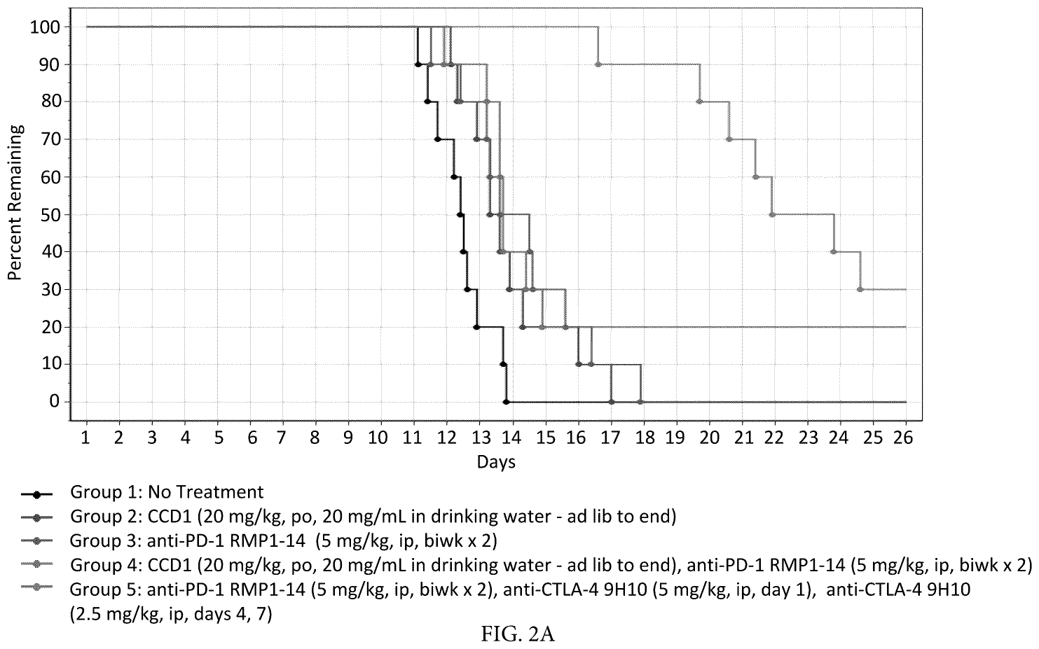

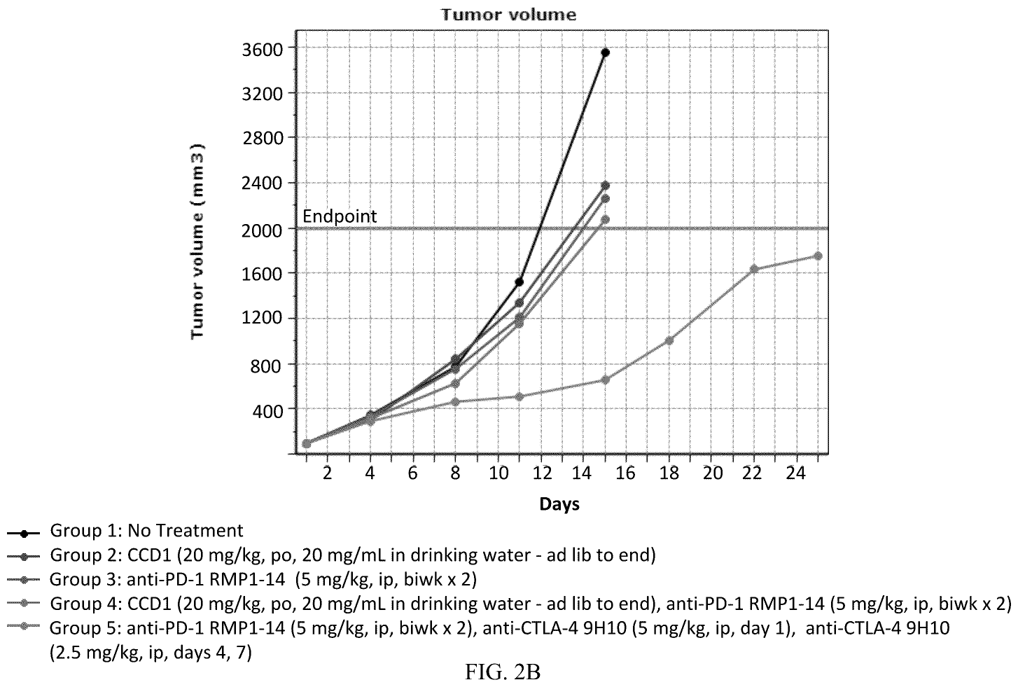

FIG. 2A shows the Kaplan-Meier survival data for all groups and FIG. 2B shows the mean tumor volume for all groups. In FIGS. 2A and 2B, CCD1 means formate.

Example 2: Modulation of T cell activation and survival by formate. Naïve CD8+ T cells were isolated from mouse spleen. Cells were activated at a cell density of 106 cells/mL using plate-bound αCD3/αCD28+100 U/mL TL2 in RPMI containing 10% FBS. The effect of addition of 1 mM formate to the media was tested. 1 mM formate enhanced size at day 1 post activation, an early measure of T cell activation, from 9.5 μm to 10.2 μm. In addition, the extent of cells showing cell surface activation markers (CD25+, CD69+) was increased from 78% to 88%. Formate also reduced the concentration of the reduced pyridine nucleotides cofactor NADH by 1.8-fold (p<0.005), a favorable change for enabling T cell function in a hypoxic tumor microenvironment. In growing CD8+ T cells, generated as above but allowed to start proliferating for several days before addition of formate, formate increased cell viability from 90% to 95% (i.e., decreased dead cells from 10% to 5%).

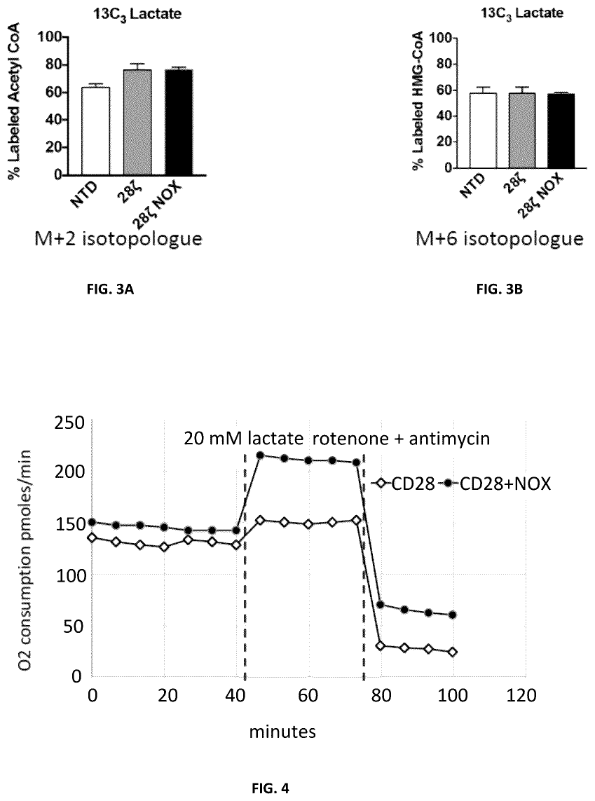

Example 3: CAR-T cells actively metabolize lactate. CAR-T cells comprising a CAR targeting mesothelin were grown in culture, without (non-transduced, NTD) or with expression of the CD28ζ or CD28ζ and NOX from Lactobacillus brevis (LbNOX) (UniProtKB Accession Number Q8KRG4). The medium composition was RPMI (10 mM glucose, 2 mM glutamine), 1 mM pen strep (penicillin streptomycin), 1 mM hepes, and 10% dialyzed serum. This medium was supplemented with 20 mM ¹³C3-lactate overnight. The contribution of lactate carbon to acetyl-CoA and HMG-CoA was measured by LC-MS. As shown in FIGS. 3A and 3B, CAR-T cells intrinsically actively take up and utilize lactate.

Example 4: NOX drives oxygen consumption and NAD production in CAR-T cells. CAR-T cells comprising a CAR targeting mesothelin were generated by activating T cells with dynabead (CD3/CD28) and then co-infecting with lentivirus for CAR as well as NADH Oxidase (cytoplasmic) from Lactobacillus brevis (LbNOX) (UniProtKB Accession Number Q8KRG4). Experiments were performed on day 10. Oxygen consumption was measured using a Seahorse extracellular flux analyzer as a function of time. This was initially performed in basal medium (XF RPMI base medium without phenol red supplemented with 5 mM glucose, 2 mM glutamine, and 0.5 mM hepes and adjusted to pH 7.4) and subsequently 20 mM lactate was added, followed by 5 μM rotenone and antimycin to block the respiratory chain. Note that rotenone and antimycin do not block oxygen consumption by NOX.

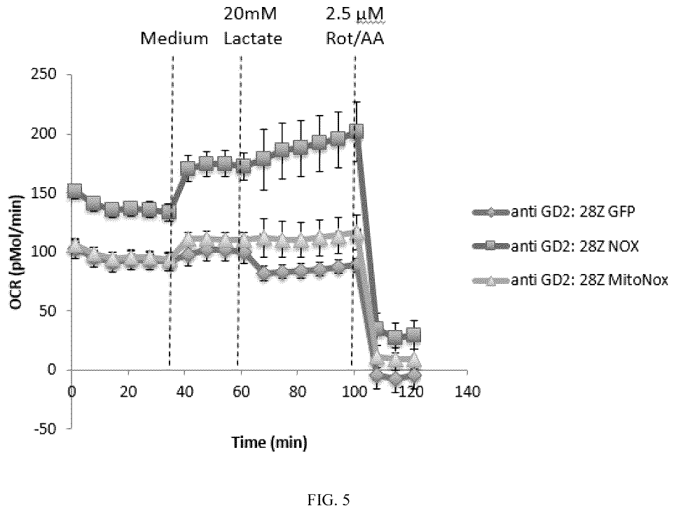

As shown in FIG. 4, cytosolic NOX expression increased the basal oxygen consumption of the CAR-T cells. This effect was magnified by addition of lactate to mimic the solid tumor microenvironment. Subsequently, upon inhibition of normal cellular respiration by rotenone+antimycin, the residual respiration was much higher in the NOX-expressing cells. This validates the effectiveness of NOX to drive oxygen consumption and NADH oxidation to restore NAD in CAR-T cells.

Similar results were obtained with CAR-T cells comprising a CAR targeting GD-2. See FIG. 5. FIG. 5 shows that cytosolic NOX expression induces basal T cell oxygen consumption and mitochondrial NOX expression supports oxygen consumption, especially in the presence of lactate.

Example 5: NOX drives oxygen consumption and NAD production in primary human T cells. T cells were induced into proliferation by dynabead (CD3/CD28) stimulation (3:1 beads/cell) and expanded in culture. NOX expression (mitochondrial or cytosolic) was induced by lentivirus (co-expressing GFP by T2A independent ribosomal entry site) in cells seeded at 2×10⁶ cells/well in 6 cell plates filled with 2 mL media. The NOX was from Lactobacillus brevis (LbNOX) (UniProtKB Accession Number Q8KRG4). Oxygen consumption was measured using a Seahorse extracellular flux analyzer. This was initially performed in basal medium (XF RPMI base medium without phenol red supplemented with 5 mM glucose, 2 mM glutamine, and 0.5 mM hepes and adjusted to pH 7.4) and subsequently 20 mM lactate was added, followed by 5 μM rotenone and antimycin to block the respiratory chain. Oxygen consumption data are presented in the table below.

| | O₂ consumption (pmole/min) | | |
|---|---|---|---|
| Condition | Control | Cyto NOX | Mito NOX |
| Basal | 58 | 120 | 70 |
| +Lactate | 86 | 250 | 140 |
| +Rotenone + Antimycin | 24 | 130 | 67 |

Cytosolic NOX expression induces basal T cell oxygen consumption. Mitochondrial NOX expression supports oxygen consumption, especially in the presence of lactate. Both mitochondrial and cytosolic NOX expression induce lactate-stimulated and rotenone/antimycin-resistant oxygen consumption.

Example 6: NOX reduces the expression of the immune checkpoint molecule Tim-3. CD8+ T cells expanded in vitro from a human donor were transfected (or not) with cytoplasmic NOX from Lactobacillus brevis (LbNOX) (UniProtKB Accession Number Q8KRG4). Cell growth and expression of immune checkpoint markers (Tim-3, PD-1, Lag-3), whose expression is undesirable for immunotherapy, were monitored in cells grown under standard normoxic conditions, in the presence of high lactate (1 mM glucose, 20 mM lactate), and in hypoxia (1% oxygen). Data were collected during the expansion phase from day 7-day 9 after T cell stimulation. NOX expression increased proliferation by about 50% under basal normoxic media conditions and in the presence of lactate. In hypoxia, however, NOX expression decreased cell count by 2-fold, reflecting the ability of NOX in the context of hypoxia to deplete available oxygen for other cellular tasks, which in the context immunotherapy (e.g., for solid tumors) will lead to oxygen depletion of other tumor cell types, including the epithelial cancer cells and stromal cells (e.g., cancer-associated fibroblasts, stellate cells, macrophages, etc.), thereby inhibiting tumor growth. PD-1 and Lag-3 expression were not altered by NOX. NOX expression desirably reduced expression of the T cell-exhaustion-related marker Tim-3 under all three conditions (basal, high lactate, and hypoxia). Notably, the increase in Tim-3 which normally occurs with transition to hypoxia was blocked by NOX expression.

Example 7. The antitumor effectiveness of T cells with or without (cytosolic and/or mitochondrial) NOX expression is compared in a tumor model, e.g., as per Wang et al. Cancer Immunology Research 2015. In one arm, a hypoxic tumor xenograft model is selected. In another arm, a less hypoxic tumor xenograft model is selected. To each animal, 7 million T cells are delivered. Experiments are conducted in 8 mice per group with T cells introduced at a tumor volume of approximately 300 mm³. Tumor volume is then recorded every few days.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

As used herein, the singular forms “a,” “an” and “the” include plural referents unless the context clearly dictates otherwise. Thus, for example, reference to “an agent” can include a plurality of agents. Further, the plurality can comprise more than one of the same agent or a plurality of different agents.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of promoting an immune response to a tumor in a subject in need thereof, comprising administering to the subject an effective amount of an agent that provides a one-carbon unit and an agent that promotes an anti-tumor response.

2. A method of treating cancer in a subject in need thereof, comprising administering to the subject an effective amount of an agent that provides a one-carbon unit and an agent that promotes an anti-tumor response.

3. A method of treating immune dysfunction in a subject in need thereof, comprising administering to the subject an effective amount of an agent that provides a one-carbon unit and an agent that promotes an immune response.

4. The method of claim 1, wherein the agent that provides a one-carbon unit is serine, glycine, histidine, tryptophan, formic acid, folic acid, 5-methyl-tetrahydrofolate, 5-formyl-tetrahydrofolate, monomethylglycine, dimethylglycine, glycine betaine, choline or glucose, a prodrug of any of the foregoing or a salt of any of the foregoing.

5. The method of claim 4, wherein the agent that provides a one-carbon unit is formic acid, a prodrug thereof or a salt of either of the foregoing.

6. The method of claim 4, wherein the agent that provides a one-carbon unit is folic acid, 5-methyltetrahydrofolate, 5-formyltetrahydrofolate, a prodrug of the foregoing or a salt of any of the foregoing.

7. The method of claim 1, further comprising administering to the subject glycine, a prodrug thereof or a salt of either of the foregoing.

8. The method of claim 1, wherein the agent that promotes an anti-tumor response is an antibody, a vaccine or a population of immune cells.

9. The method of claim 1, wherein the agent that promotes an anti-tumor response is an agent that inhibits PD-1, PD-L1 or CTLA-4.

10. The method of claim 9, wherein the agent that promotes an antitumor response is an antibody that inhibits PD-1, PD-L1 or CTLA-4.

11. The method ofany one of claim 1, wherein the subject has cancer.

12. The method of claim 1, wherein the subject has lung cancer.

13. The method of claim 1, wherein the subject has a solid tumor.

14. The method of claim 1, wherein the subject is an aged human.

15.-82. (canceled)

83. The method of claim 1, wherein the agent that promotes an anti-tumor response is an agent that inhibits PD-1.

84. The method of claim 83, wherein the agent that promotes an anti-tumor response is an antibody that inhibits PD-1.

85. The method of claim 1, wherein the agent that promotes an anti-tumor response is a checkpoint inhibitor.

86. The method of claim 4, wherein the prodrug is an ester comprising a hydrolyzable group.

87. The method of claim 86, wherein the hydrolyzable group is selected from the group consisting of methyl, ethyl, n-propyl, isopropyl, n-butyl, sec-butyl, isobutyl, tert-butyl, pentyl, hexyl, heptyl, allyl, ethoxymethyl, methoxyethyl, methoxyethoxymethyl, methoxyethoxyethyl, benzyl, pentafluorophenyl, 2-N-(morpoholino)ethyl, dimethylaminoethyl and para-methoxybenzyl.

88. The method of claim 86, wherein the hydrolyzable group is (C₁-C₁₀)alkyl.
